(19) United States
(12) Patent Application Publication
(10) Pub. No.: US 2009/0024095 A1
(43) Pub. Date: Jan. 22, 2009

(54) Syringe Designed to Be Pre-Filled Then Sterilized by Steam Autoclaving

(75) Inventor: Pierre Frezza, Charly (FR)

Correspondence Address: OLIFF & BERRIDGE, PLC, P.O. BOX 320850, ALEXANDRIA, VA, 22320-4850, US

(73) Assignee: LABORATOIRE AGUETTANT, LYON (FR)

(21) Appl. No.: 11/989,226

(22) PCT Filed: Aug. 30, 2006
(86) PCT No.: PCT/FR2006/002007

§ 371 Date: Apr. 18, 2008

(30) Foreign Application Priority Data
Sep. 2, 2005 (FR) 0509011

(51) Int. Cl. A61M 5/315

(52) U.S. Cl. 604/222

(57) ABSTRACT

The invention concerns a syringe comprising: a body including a cylindrical side wall having an open upstream end with an inner annular rim and a downstream end closed by a transverse wall provided with an orifice; a shaft equipped with a plunger having at least two annular sealing lips between which is defined at least one annular chamber. The shaft, which can slide inside the body, defines inside the body an inner chamber designed to be at least partly filled with a content. Delivery passages provided inside the body are configured to communication said annular chamber of the plunger with the outside of the body or with the inner chamber of the body, when the plunger is abutting against the annular rim.

The present invention relates to a syringe, and in particular a syringe designed to be pre-filled then sterilized by steam autoclaving, as well as to an assembly comprising a package and such a syringe enclosed in this package.

### BACKGROUND OF THE INVENTION

A “pre-filled” syringe is a ready-to-use non-reusable product. This type of syringe is filled with the desired liquid and sterilized industrially by the pharmaceutical laboratory. Sterility of the container/contents assembly is achieved either by filling under aseptic conditions syringes whose components have been pre-sterilized, or by steam-sterilizing the container/contents assembly at the end. The present invention is concerned with the latter method of sterilization only.

### DESCRIPTION OF THE PRIOR ART

A pre-filled syringe of the prior art is shown in longitudinal cross section in FIG. 1.

The syringe 100 comprises a cylindrical body 101 whose downstream end is closed by a wall 102 containing a “Luer” or “Luer-lock” type access 103 which in turn is sealed by a removable cap 104. The upstream end of the body 101 is open to permit the forcible insertion of a rod 105, forming a plunger, fitted with a piston 106 forming a seal, the latter generally comprising three sealing lips 107 defining two sealed annular chambers 108, 109.

The rod 105/piston 106 assembly can slide leak-tightly inside the body 101 when pushed by for example a nurse, causing the liquid 110 contained inside the body 101 to exit toward a patient via the access 103.

Lastly, the upstream end of the body 101 comprises an annular bead 111 designed to act as an end stop to the rod 105/piston 106 assembly and prevent it escaping from the body 101.

The industrial process of filling and sterilizing the syringe 100 is as follows: after putting on the cap 104, filling, inserting the piston 106 and inserting and screwing the rod 105 onto the piston 106, the syringe 100 is enclosed in a package (blister pack) comprising a thermoformed plastic part closed by a peel-off paper seal. This paper possesses the property of being permeable to water vapor but largely impassable to microorganisms.

For steam sterilization to meet regulatory requirements (guaranteed sterility even in the event of heavy initial contamination), it must be carried out at a temperature of at least 121° C. for at least 15 minutes in so-called “wet” heat, which means that all parts requiring sterilization must be in contact with the steam, whether the steam arrives from the autoclave chamber after passing through the paper seal of the package, or whether it is from the vaporized contents of the syringe.

However, the annular chambers 108, 109 formed between the lips 107 of the piston 106 are sealed off and are not therefore accessible to the steam. Sterilization will therefore be by “dry” heat. As a result, in order to sterilize these annular chambers, the sterilization period must be much longer than in “wet” heat (about 60 minutes instead of 15 minutes). This extended sterilization cycle causes three major problems:

the production cost is much higher;
there is increased breakdown of the material of the syringe body, increasing the risk of release of breakdown products (plastic additives or components of the glass such as aluminum) in the liquid 110;
it is impossible to sterilize certain active principles at 121° C. for 60 minutes because they would suffer unacceptable breakdown.

It is an object of the present invention to overcome the above problems by simple means without greatly increasing the cost of the syringe.

### SUMMARY OF THE INVENTION

To this end, and in accordance with a first aspect, the invention relates to a syringe comprising:
a body comprising a generally cylindrical side wall having an open upstream end in the vicinity of which an inward annular bead is formed, and a downstream end closed by a transverse wall containing an orifice;
a rod with a piston at one end forming a seal and possessing at least two annular sealing lips between which at least one annular chamber is defined, said rod being designed to be inserted into and slid along the inside of the body.

An inner chamber designed to be at least partly filled with contents is then defined between the piston and the transverse wall of the body.

In accordance with a general definition of the invention, the syringe also comprises means of communication formed in the body of the syringe and designed to place said annular chamber or chambers of the piston in communication with the outside of the body or with the inner chamber of the body, when the piston is inside the body and in contact with the annular bead.

In practice, in the storage position (in other words when the syringe is pre-filled and is at storage temperature), the means of communication are situated upstream of the piston and there is no risk to the integrity of the contents of the inner chamber of the syringe body.

During sterilization, the piston is in contact with the bead because of the pressure inside the body due to the syringe contents entering the vapor phase. The means of communication allow the steam to gain access to the annular chambers of the piston, either entering from outside the body (meaning from the chamber of the autoclave in which sterilization is being carried out) or from inside the body itself, in other words from contents that have entered the vapor phase. Of course, one chamber cannot be in communication at the same time with the outside of the body and with the inner chamber if the integrity of the inner chamber is not to be put at risk.

The invention therefore enables sterilization by wet heat between the lips of the syringe piston, and as a result greatly reduces the amount of heat required for sterilization.

Advantageously, the means of communication are also designed to place at least one region of the outside face of at least one sealing lip in communication with the outside of the body or with the inner chamber of the body, when the piston is in contact with the annular bead, while at least one other sealing lip continues, when the piston is in this position, to seal off the inner chamber of the body.

The expression “integrity of the inner chamber” here means that no product has entered said inner chamber and none of the contents have leaked out of the body of the syringe (the contents may however reach the annular chambers of the piston).

Throughout the description, the terms “upstream” and “downstream” will be used with reference to the direction of flow of the contents on their way out of the syringe, when the syringe is in use.

In a first embodiment, the means of communication comprise at least one groove formed approximately axially in the side wall of the cylindrical body, on its inside face, which groove leads out of the body at the upstream end of the body and extends as far as a downstream end situated, when the piston is in contact with the inward annular bead, upstream of the downstream sealing lip and downstream of that sealing lip which is situated immediately upstream of the downstream sealing lip, the radial depth of the groove being great enough to locally break the seal between the outside face of at least the upstream sealing lip and the inside face of the side wall of the body.

In a second embodiment, the means of communication comprise at least one orifice formed in the side wall of the body and designed to place the annular chamber or chambers of the piston in communication with the outside of the body when the piston is in contact with the annular bead, the downstream edge of said orifice being situated, when the piston is in contact with the inward annular bead, upstream of the downstream sealing lip and downstream of that sealing lip which is situated immediately upstream of the downstream sealing lip.

In a third embodiment, the means of communication comprise at least one slot formed in the side wall of the cylindrical body, in its inside face, said slot extending between:
an upstream end situated, when the piston is in contact with the inward annular bead, downstream of the upstream sealing lip and upstream of that sealing lip which is situated immediately downstream of the upstream sealing lip;
and a downstream end situated, when the piston is in contact with the inward annular bead, downstream of the downstream sealing lip;

the axial length of the slot being less than the total axial length of the piston, and the radial depth of the slot being great enough to locally break the seal between the outside face of at least the downstream sealing lip and the inside face of the side wall of the body.

The slot may for example be annular, of the same axis as the cylindrical side wall of the body.

In one possible embodiment, the piston possesses three annular sealing lips defining two separate annular chambers, the means of communication being designed to place each of the annular chambers in communication with the outside of the body or with the inner chamber of the body when the piston is inside the body and in contact with the annular bead.

Lastly, in accordance with a second aspect, the invention relates to an assembly comprising on the one hand an essentially bacteria-proof package, of which at least a part is water vapor-permeable, and on the other hand a syringe as described above, said syringe being enclosed in said package and its inner chamber at least partly filled with contents.

### BRIEF DESCRIPTION OF THE DRAWINGS

There now follows a description of a number of possible embodiments of the invention, as non-restrictive examples, with reference to the accompanying figures:

FIG. 1 is a longitudinal section through a prior art syringe;

FIGS. 2 and 3 are partial views in longitudinal section of a pre-filled syringe in a first embodiment of the invention, during storage and during sterilization, respectively;

FIGS. 4 and 5 are views similar to FIGS. 2 and 3, showing a second embodiment; and

FIGS. 6 and 7 are views similar to FIGS. 2 and 3, showing a third embodiment, in which the body of the syringe is shown in its entirety.

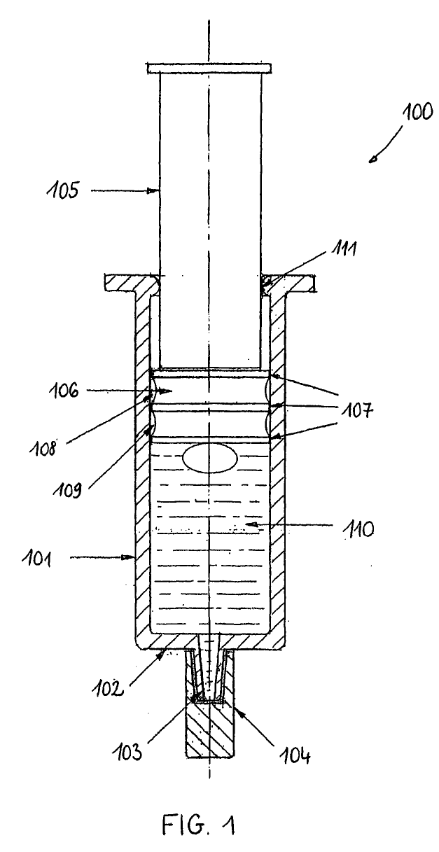

### DESCRIPTION OF THE PREFERRED EMBODIMENTS

A syringe 1 comprises in the first place a body 2 comprising a generally cylindrical side wall 3 of axis 4. The side wall 3 has an upstream end which is open and a downstream end which is closed by a transverse wall 5 containing an orifice 6 and extended by a conical nozzle 7 of the “Luer” or “Luer-lock” type.

At its upstream end, the body has both a collar 8 for a nurse to press against with the fingers, and an inward annular bead 9.

The syringe 1 also comprises a rod 10 forming a plunger, at the downstream end of which is a piston 11. The piston 11 possesses three annular sealing lips, namely an upstream lip 12, an intermediate lip 13 and a downstream lip 14, designed to be in contact with the inside face 15 of the side wall 3 of the body 2. An annular chamber is defined between each two successive lips. In the embodiment illustrated, the piston 11 therefore has two annular chambers 16, 17.

The syringe 1 (body and rod) is here made of plastic, but it could be of glass.

The rod 10 is designed to be inserted into the body 2 and slide along inside it leak-tightly when pushed by a user. The piston 11 and the inside of the body 2 are generally coated with silicone so that the piston slides easily.

In this way an inner chamber is defined inside the body 2, between the transverse wall 5 and the piston 11. The inner chamber is filled with contents 18 which may be a medicinal solution, a solvent, etc. There is also usually a gas bubble 19 (air or nitrogen, for example, depending on the case) left inside this inner chamber.

Lastly, the syringe 1 comprises a removable cap 20 for closing the orifice 6 formed in the transverse wall 5 of the body 2.

The syringe 1, prefilled and equipped with the rod 10 and cap 20 is put in a package of the type described earlier. The whole is then placed in an autoclave for steam sterilization of the syringe 1.

According to the invention, means of communication are formed in the body 2 of the syringe 1 to allow the steam to sterilize the annular chambers 16, 17 of the piston 11.

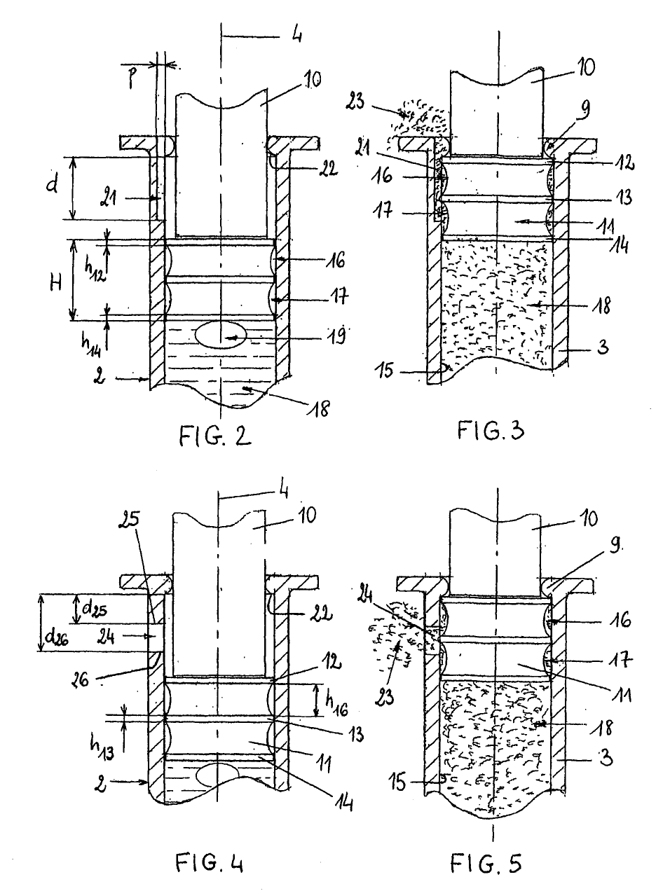

In a first embodiment, shown in FIGS. 2 and 3, the means of communication consist of at least one groove 21 formed essentially axially in the side wall 3 of the body 2, from the inside face 15. The groove 21 preferably leads out of the body 2 at the upstream end of the body, interrupting the bead 9, locally. In a variant, the groove or grooves 21 need not lead out of the body but could have an upstream end situated close to the downstream face 22 of the bead 9.

The groove 21, or each groove 21 has the following features:
the axial distance d between the downstream face 22 of the bead 9 and the downstream end of the groove 21 is such that:

d>h₁₂+h₁₆+h₁₃ and d<H−h₁₄
where
   H is the total axial length of piston 11,
   h₁₂, h₁₃ and h₁₄ are the axial lengths of the upstream 12, intermediate 13 and downstream 14 sealing lips, respectively, of the piston 11,
   h₁₆ is the axial length of the upstream annular chamber 16 of the piston 11;
the radial depth p of the groove 21 is great enough to locally break the seal between the outer face of the upstream 12 and intermediate 13 sealing lips and the inside face 15 of the side wall 3 of the body 2.

FIG. 2 shows the syringe 1 in the storage position (syringe 1 at room temperature, for example in its package). The capacity of the body 2 is adapted to suit the desired volume of the contents 18 so that, in this position, the piston 11 is situated downstream of the groove 21. In this way the contents 18 (in the liquid phase) of the syringe 1 are isolated by the three lips 12, 13, 14 of the piston 11. The chambers 16 and 17 are sealed off and the groove 21 has no function.

At the start of the sterilization cycle, the syringe 1 in its package is placed in the autoclave chamber, at room temperature, and autoclave pressure is established. The contents 18 of the syringe are in the liquid phase, so there is no pressure on the piston 11 to push it out of body 2 of the syringe 1. In any case, the pressure in the autoclave chamber acts on the rod 10 and tends to push the piston 11 into the body 2 of the syringe 1. The piston 11 is therefore always in a position such as to isolate the contents 18.

The temperature in the autoclave chamber rises gradually to 121° C., with an absolute pressure of around 2 bar. The contents 18 of the syringe 1 now vaporize, thus generating pressure inside the body 2. This pressure is proportional to the temperature of the steam, and also varies as a function of the amount of gas (the bubble 19) in the body 2 of the syringe 1.

When the pressure in the body 2 of the syringe 1 is generating a force greater than that exerted by the autoclave pressure on the rod 10, added to the force required to make the piston 11 slide, the piston retreats until it contacts the bead 9 (FIG. 3). The steam 23 present in the autoclave chamber will now enter the groove 21. Given the dimensional relationships mentioned above, the steam 23 also passes into the annular chambers 16, 17, thereby sterilizing these chambers with so-called wet heat. In this position, the contents 18 of the syringe 1 are sealed off by the upstream lip 14 of the piston 11, because the groove 21 has sufficient length to enable the two chambers 16, 17 to communicate with the outside of the body 2, and is sufficiently short for there to be no risk of contamination of the inner chamber.

Clearly, the dimensions of the body 2 of the syringe 1 and the volume of the contents 18 are chosen so that, during sterilization, the piston 11 makes firm contact with the bead 9, and is therefore positioned correctly relative to the groove 21. Furthermore, the near incompressibility of the piston 11 ensures that the contents 18 remain sealed off because the upstream lip 14 stays at a distance from the groove 21.

At the end of the sterilization cycle (in the cooling phase), the pressure in the body 2 of the syringe 1 will gradually drop and the contents 18 of the syringe 1 will return to the liquid state. When the pressure in the autoclave chamber generates a force greater than that generated by the contents of the syringe 1 added to that necessary to make the piston 11 slide, the latter will move back along the body 2 of the syringe 1 to its initial position (FIG. 2).

In a second embodiment, shown in FIGS. 4 and 5, the means of communication consist of at least one orifice 24 formed in the side wall 3 of the body 2. The orifice 24, which is preferably circular and radial, has an upstream edge 25 and a downstream edge 26: these are situated at distances d₂₅ and d₂₆, respectively, from the downstream face 22 of the bead 9, such that:

d₂₅>h₁₂ and d₂₅<h₁₂+h₁₆

d₂₆>h₁₂+h₁₆+h₁₃ and d₂₆<H−h₁₄.

Once again the dimensions of the body 2 are adapted to the volume of the contents 18 so that, in the storage position (FIG. 4), the piston 11 is at a distance from the orifice 24, so that the seal of the inner chamber is not affected by the orifice 24. However, the orifice 24 is designed to place the two annular chambers 16, 17 in communication with the outside of the body 2, in order to allow steam 23 to enter during sterilization (FIG. 5), when the piston 11 is in contact with the bead 9.

Finally, in a third embodiment, the means of communication are an annular slot 27 formed in the side wall 3 of the body 2 from the inside face 15. This slot 27 has an upstream end 28 and a downstream end 29, and has the following features:
the axial distance d₂₈ between the downstream face 22 of the bead 9 and the upstream end 28 of the slot 27 is such that:

d₂₈>h₁₂ and d₂₈<h₁₂+h₁₆;
the axial distance d₂₉ between the downstream face 22 of the bead 9 and the downstream end 29 of the slot 27 is such that: d₂₉>H;
the radial depth p′ of the slot 27 is great enough to locally break the seal between the outer face of the intermediate 13 and downstream 14 sealing lips and the inside face 15 of the side wall 3 of the body 2; and
the axial length of the slot (d₂₉−d₂₈) is less than the total axial length H of the piston 11.

This last feature ensures that the inner chamber is sealed off from the outside of the body 2 of the syringe 1 whatever the position of the piston 11 in the body 2, between the storage position and the position of contact with the bead 9.

In a variant the slot 27 may occupy only a fraction of the perimeter of the body 2.

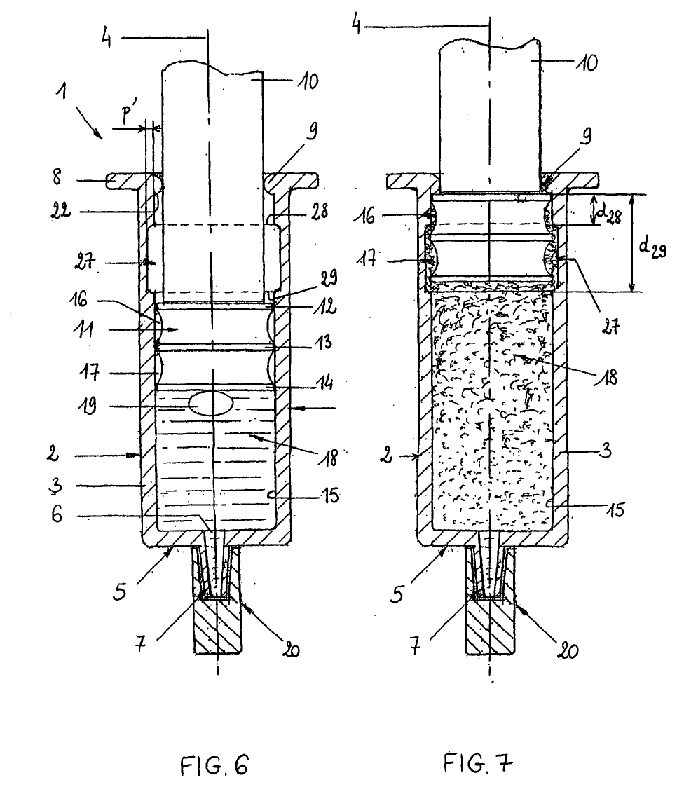

As in the embodiments described above, the body 2 is designed on the basis of the volume of the contents 18 so that the piston 11 is situated at a distance from the slot 27 when in the storage position (FIG. 6): the integrity of the inner chamber is therefore not affected by the slot 27.

During sterilization (FIG. 7), the piston 11 is pushed against the bead 9, and the slot 27 therefore places the inside of the body 2 in communication with the annular chambers 16, 17. In this embodiment, the steam with which the annular chambers 16, 17 of the piston 11 are sterilized is formed by the contents 18, in the gas phase, of the body 2 of the syringe 1, rather than by steam from the autoclave chamber. One of the advantages of this embodiment is that it enables the downstream lip 14 of the piston 11 to be sterilized.

Thus, by adding means of steam communication situated upstream of the piston when the syringe is in the storage position and surrounding the annular chambers of the piston during sterilization, the invention enables steam to enter between the lips of the piston while maintaining the isolation of the syringe contents from the steam present in the autoclave chamber.

It goes without saying that the invention is not limited to the embodiments described above by way of examples but that on the contrary it encompasses all variants. In particular, the means of communication could take the form of a suitable combination of the three individual embodiments that have been described.

1. A syringe comprising:
a body comprising a generally cylindrical side wall having an open upstream end in the vicinity of which an inward annular bead is formed, and a downstream end closed by a transverse wall containing an orifice;
a rod forming a plunger, with a piston at one end forming a seal possessing at least two annular sealing lips between which at least one annular chamber is defined, said rod being designed to be inserted into and slid along the inside of the body;
an inner chamber designed to be at least partly filled with contents and then being defined between the piston and the transverse wall of the body;
said syringe being wherein it also comprises means of communication formed in the body of the syringe and designed to place said annular chamber or chambers of the piston in communication with the outside of the body or with the inner chamber of the body, when the piston is inside the body and in contact with the annular bead.

2. The syringe as claimed in claim 1, wherein the means of communication are also designed to place at least one region of the outside face of at least one sealing lip in communication with the outside of the body or with the inner chamber of the body, when the piston is in contact with the annular bead, while at least one other sealing lip continues, when the piston is in this position, to seal off the inner chamber of the body.

3. The syringe as claimed in claim 1, wherein the means of communication comprise at least one groove formed essentially axially in the side wall of the cylindrical body, on its inside face, which groove leads out of the body at the upstream end of the body and extends as far as a downstream end situated, when the piston is in contact with the inward annular bead, upstream of the downstream sealing lip and downstream of that sealing lip which is situated immediately upstream of the downstream sealing lip, the radial depth of the groove being great enough to locally break the seal between the outside face of at least the upstream sealing lip and the inside face of the side wall of the body.

4. The syringe as claimed in claim 1, wherein the means of communication comprise at least one orifice formed in the side wall of the body and designed to place the annular chamber or chambers of the piston in communication with the outside of the body when the piston is in contact with the annular bead, the downstream edge of said orifice being situated, when the piston is in contact with the inward annular bead, upstream of the downstream sealing lip and downstream of that sealing lip which is situated immediately upstream of the downstream sealing lip.

5. The syringe as claimed in claim 1, wherein the means of communication comprise at least one slot formed in the side wall of the cylindrical body, in its inside face, said slot extending between:
an upstream end situated, when the piston is in contact with the inward annular bead, downstream of the upstream sealing lip and upstream of that sealing lip which is situated immediately downstream of the upstream sealing lip;
and a downstream end situated, when the piston is in contact with the inward annular bead, downstream of the downstream sealing lip;
the axial length of the slot being less than the total axial length of the piston, and the radial depth of the slot being great enough to locally break the seal between the outside face of at least the downstream sealing lip and the inside face of the side wall of the body.

6. The syringe as claimed in claim 5, wherein the slot is annular and has the same axis as the cylindrical side wall of the body.

7. The syringe as claimed in claim 1, wherein the piston possesses three annular sealing lips defining two separate annular chambers, the means of communication being designed to place each of the annular chambers in communication with the outside of the body or with the inner chamber of the body when the piston is inside the body and in contact with the annular bead.

8. An assembly comprising on the one hand an essentially bacteria-proof package, of which at least a part is water vapor-permeable, and on the other hand a syringe as claimed in claim 1, said syringe being enclosed in said package and its inner chamber at least partly filled with contents.

9. The syringe as claimed in claim 2, wherein the means of communication comprise at least one groove formed essentially axially in the side wall of the cylindrical body, on its inside face, which groove leads out of the body at the upstream end of the body and extends as far as a downstream end situated, when the piston is in contact with the inward annular bead, upstream of the downstream sealing lip and downstream of that sealing lip which is situated immediately upstream of the downstream sealing lip, the radial depth of the groove being great enough to locally break the seal between the outside face of at least the upstream sealing lip and the inside face of the side wall of the body.

10. The syringe as claimed in claim 2, wherein the means of communication comprise at least one orifice formed in the side wall of the body and designed to place the annular chamber or chambers of the piston in communication with the outside of the body when the piston is in contact with the annular bead, the downstream edge of said orifice being situated, when the piston is in contact with the inward annular bead, upstream of the downstream sealing lip and downstream of that sealing lip which is situated immediately upstream of the downstream sealing lip.

11. The syringe as claimed in claim 2, wherein the means of communication comprise at least one slot formed in the side wall of the cylindrical body, in its inside face, said slot extending between:
an upstream end situated, when the piston is in contact with the inward annular bead, downstream of the upstream sealing lip and upstream of that sealing lip which is situated immediately downstream of the upstream sealing lip;
and a downstream end situated, when the piston is in contact with the inward annular bead, downstream of the downstream sealing lip;
the axial length of the slot being less than the total axial length of the piston, and the radial depth of the slot being great enough to locally break the seal between the outside face of at least the downstream sealing lip and the inside face of the side wall of the body.

12. The syringe as claimed in claim 2, wherein the piston possesses three annular sealing lips defining two separate annular chambers, the means of communication being designed to place each of the annular chambers in communication with the outside of the body or with the inner chamber of the body when the piston is inside the body and in contact with the annular bead.

13. The syringe as claimed in claim 3, wherein the piston possesses three annular sealing lips defining two separate annular chambers, the means of communication being designed to place each of the annular chambers in communication with the outside of the body or with the inner chamber of the body when the piston is inside the body and in contact with the annular bead.

14. The syringe as claimed in claim 4, wherein the piston possesses three annular sealing lips defining two separate annular chambers, the means of communication being designed to place each of the annular chambers in communication with the outside of the body or with the inner chamber of the body when the piston is inside the body and in contact with the annular bead.

15. The syringe as claimed in claim 5, wherein the piston possesses three annular sealing lips defining two separate annular chambers, the means of communication being designed to place each of the annular chambers in communication with the outside of the body or with the inner chamber of the body when the piston is inside the body and in contact with the annular bead.

16. The syringe as claimed in claim 6, wherein the piston possesses three annular sealing lips defining two separate annular chambers, the means of communication being designed to place each of the annular chambers in communication with the outside of the body or with the inner chamber of the body when the piston is inside the body and in contact with the annular bead.

17. The syringe as claimed in claim 9, wherein the piston possesses three annular sealing lips defining two separate annular chambers, the means of communication being designed to place each of the annular chambers in communication with the outside of the body or with the inner chamber of the body when the piston is inside the body and in contact with the annular bead.

18. The syringe as claimed in claim 10, wherein the piston possesses three annular sealing lips defining two separate annular chambers, the means of communication being designed to place each of the annular chambers in communication with the outside of the body or with the inner chamber of the body when the piston is inside the body and in contact with the annular bead.

19. The syringe as claimed in claim 11, wherein the piston possesses three annular sealing lips defining two separate annular chambers, the means of communication being designed to place each of the annular chambers in communication with the outside of the body or with the inner chamber of the body when the piston is inside the body and in contact with the annular bead.
